(19) United States
(12) Patent Application Publication
(10) Pub. No.: US 2023/0241453 A1
(43) Pub. Date: Aug. 3, 2023

(54) EXERCISE MOTION SYSTEM AND METHOD

(71) Applicant: Activarium, LLC, Excelsior, MN (US)

(72) Inventors: Elizabeth Lowery, Excelsior, MN (US); Nelson Mane, Odessa, FL (US); Mark Randel, Flower Mound, TX (US); Marvin Douma, Dallas, TX (US)

(21) Appl. No.: 18/133,854

(22) Filed: Apr. 12, 2023

Related U.S. Application Data
(63) Continuation-in-part of application No. 16/220,078, filed on Dec. 14, 2018, now abandoned.
Application No. 16/220,078 is a continuation-in-part of application No. 14/920,840, filed on Oct. 22, 2015, now abandoned.
(60) Provisional application No. 62/067,166, filed on Oct. 22, 2014.

(51) Int. Cl. A63B 24/00; A63F 13/211; A63F 13/212; A63F 13/214; A63B 71/06

(52) U.S. Cl. CPC A63B 24/0006; A63F 13/211; A63F 13/212; A63F 13/214; A63B 71/0622; A63B 2024/0012; A63B 2220/803

(57) ABSTRACT

An exercise therapy video game and monitor system for physical targeted activation and cognitive skills for learning fluency of an individual. More specifically, the system includes a whole-body fitness program that integrates motion, motor-sensory learning, and vision using games and motion sensing technology to track video-directed body position and movements of the individual. The system includes at least a first gaming platform, interactive exercise software to provide movement instructions to the individual, a computing system, and one or more sensors, such as motion sensors or sensors associated with a mat, to track body positions and movements of the individual. The system can then provide instant feedback to improve the body positions and movements of the individual to better replicate video directions.

### CROSS-REFERENCE TO RELATED APPLICATION

This application is a continuation-in-part of U.S. Nonprovisional patent application Ser. No. 16/220,078, filed on Dec. 14, 2018, title EXERCISE MOTION SYSTEM AND METHOD, which was an application for a continuation-in-part of U.S. Nonprovisional patent application Ser. No. 14/920,840, filed on Oct. 22, 2015, titled LEARNING DEVICE, SYSTEM, AND METHOD, which claims the benefit of U.S. Provisional Patent Application No. 62/067,166, filed on Oct. 22, 2014, titled LEARNING DEVICE, SYSTEM, AND METHOD.

### FIELD OF THE DISCLOSURE

The disclosed invention relates to an exercise motion system and method for physical targeted activation and cognitive skills for learning fluency in individuals, such as children and adults. More specifically, the disclosed system and method relate to a whole-body fitness program that integrates motion, motor-sensory learning, and vision using games and motion-sensing technology to track video-directed body movements of the individual. The system provides instant feedback to improve body movements of the individual to increase the rate of neuroplasticity through enhanced replication of the exercise directions.

### BACKGROUND OF THE INVENTION

Motion-sensing video games have become increasingly popular since home video game consoles have gotten more advanced in the 21st century. While many gaming consoles have corresponding controllers that the console uses to detect the user, advancements in the field of gaming have enabled gaming consoles to substitute controllers for motion-sensing devices that can directly detect the movement of individuals. A popular motion-sensing device commonly paired with gaming consoles is Microsoft's Kinect®. Additional advancements have eliminated the need for a gaming console and allow the motion-sensing device to interact directly with a computing device or to house a computing device in the same housing, such as an Orbbec® 3D or VicoVR® device.

Additionally, individuals with cognitive mental and physical weaknesses benefit from physical exercise, and the above-mentioned gaming consoles have, due to their ability to be accessed more readily, they have become popular ways to provide physical therapy to individuals who require physical targeted activation and cognitive skills improvement. However, while broad movements have typically been detectable by these motion-sensing devices and provided as raw data to corresponding computing systems for analysis and interpretation, those computing systems have been limited in their ability to detect fine motor movements or points during which body parts first make contact with each other or overlap. These fine motor movements and body contact/overlap movements are often very useful in therapeutic situations. Therefore, a new system is needed that can receive and analyze raw data from a motion-sensing device to determine if an individual is closely and accurately mimicking or replicating fine motor and/or body contact/overlap movement instructions provided by the system on a screen.

### SUMMARY OF THE INVENTION

The disclosed system includes devices and methods that provide improved and automated therapeutic exercises for an individual. More specifically, the system includes an exercise therapy video game and monitor system for physical targeted activation and cognitive skills improvement for learning fluency a user comprising: interactive exercise software designed to provide visual movement instructions to the user via a first gaming platform having a screen; a motion sensing device configured to track the body position and movements of the user and create and output raw data corresponding to the user's body positions and movements when the user is replicating the movements of the visual movements instructions; and a computing system networked to the interactive exercise software that is configured to input the raw data and evaluate the accuracy of the user's body positions and movements, and provide a score as instant feedback to the user by causing a comparison of the user's body positions and movements to the visual movement instructions to be displayed on a screen, wherein the feedback can improve the body positions and movements of the user. In one embodiment, the computing system has been improved to incorporate software that enables the computing system to distinguish two body parts when they are in contact with each other based on tracking of the positions and movements of the two body parts by the motion sensing device. In one embodiment, the system also includes Bluetooth-enabled glasses that coordinate music play when a user is completing an exercise on the gaming platform.

Further embodiments can provide that the first body part is a user's hand and the second body part is a user's knee. Additional embodiments, can include a third body part as a user's elbow and the fourth body part as the user's ankle. In variations of these embodiments the user's hand can be a right hand and the user's knee and be a left knee; while the user's elbow can be a right elbow and the user's ankle can be a left ankle. The handedness of the body parts may also be vise-versa but maintain their opposition.

Additional embodiments may provide that the first body part is a user's finger and the second body part is a user's nose. Other embodiments may provide a determination by the computing system that first and second body parts do not appear to in the same position in space.

Some embodiments may include a second visual instruction that can be a comparison of the received raw data associated with motion and position of each of the respective body parts compared to a desired user-executed outcome of the visual movement instructions, where the system further provides a score derived from the comparison.

In some embodiments the system can calculate a score by analyzing the raw data to determine an executed outcome for the body positions and movements of the user after a predetermined amount of time, wherein the executed outcome is the position of the first body part and the second body part when the position of the first body part was closest to the position of the second body part in space; calculating a distance between the first body part position and the second body part position when they were closest to each other; comparing the distance between the first body part position and the second body part position when they were closest to each other to a pre-determined accuracy score; displaying the accuracy score comparison on the screen; and updating the second visual movement instruction based on the accuracy score comparison.

In a further embodiment the score can be 100 if there is a determination that the first and second body parts were in contact with each other. In another embodiment, the user may be able to obtain at at least three scores for the second visual movement instruction above the predetermined threshold score, the level of difficulty is increased to the next higher level. Additional embodiments may provide accuracy score that may be less than a pre-determined threshold when the computing system determines that the first body part has not made contact with the second body part.

Other embodiments may comprise a second gaming platform networked to the interactive exercise software and the computing system. Such embodiments, may also include a foot pressure sensitive device having a plurality of sensors that track foot positions and movements of the user and create raw data. These embodiments may also provide the second gaming platform outputs raw data to the computing system, and the computing system may be able to combine the raw data from the motion sensing device and the raw data from the second gaming platform to create a combined raw data set. In such an embodiment, the computing system may be able to use the combined raw data set to evaluate the accuracy of the user's body and foot positions and movements; and provide feedback to the user by displaying a comparison of the user's body and foot positions and movements to the visual movement instructions on the screen.

In an additional embodiment, an exercise therapy video game and monitor system for physical targeted activation and improvement of cognitive skills of a user, may include a system comprising: a first gaming platform that can be networked to an interactive exercise software and may be configured to provide movement instructions to the user on a screen; a motion sensing device that may include at least one sensor which can be configured to track fine-body positions and movements of the user and at least one processor that can be configured to receive raw data from the at least one sensor and may determine, corresponding to the fine-body positions and movements of the user, when the user can attempt replicating the movements of an assessment movement instruction displayed on the screen; and a computing system that can be networked to the interactive exercise software and configured to: input the received raw data and evaluate the accuracy of the fine-body positions and movements of the user that may be attempting to replicate the movements of the assessment movement instruction, wherein values can be generated that corresponds to the fine-body positions and movements of the user; while providing a training movement instruction that may have a threshold level of difficulty, which can be based on the generated values associated with the fine-body positions and movements of the user and the assessment movement instruction, on the screen, wherein the training movement instructions direct the user to put a first body part of the user in contact with a second body part of the user; sense motion with the motion sensing device, and send motion data to the computing system for at least the position and movement associated with at least one of the first body part, the second body part, a third body part, and a fourth body part; while the system may also determine a value for velocity and acceleration associated with the at least one of the first, second, third, and fourth body parts based on the motion data; and the system may confirm that the first body part is in contact with the second body part when the motion data associated with a first body part position and a second body part position indicates the first body part and the second body part are at least partially in the same position in space, and wherein a location value is assigned to the first and second body part location; determine that the first, second, third, and fourth body parts ceased moving toward each other at approximately a same point in time; display a score, based on the positions at a single point in time, of at least one of the first, second, third, and fourth body parts respectively, the velocity value, and the location value; and assign a level of difficulty above or below the threshold level of difficulty for the training movement instruction based on the score.

In further embodiments, the interactive exercise software may further instructs the user to replicate the training movement instruction at least three times where the score is greater than the threshold score for the level of difficulty. And in an additional embodiment, the interactive exercise software may further instructs the user to replicate the training movement instruction of a higher level of difficulty once the user has received at least three scores higher than the threshold score of the current level of difficulty.

The Summary of the Invention is neither intended nor should it be construed as being representative of the full extent and scope of the present invention. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description of the Invention, and no limitation as to the scope of the present invention is intended by either the inclusion or non-inclusion of elements, components, etc. in this Summary of the Invention. Additional aspects of the present invention will become more readily apparent from the Detail Description, particularly when taken together with the drawings.

### BRIEF DESCRIPTION OF THE DRAWINGS

FIG. 1 illustrates various cognitive brain functions impacted by physical targeted activation.

FIG. 2 illustrates a proposed whole-system therapy program.

FIG. 3 illustrates a first set of exercises for the disclosed system according to one embodiment.

FIG. 4 illustrates a second set of exercises for the disclosed system according to one embodiment.

FIG. 5 illustrates a display showing activities used to stimulate a user's left hemisphere according to one embodiment.

FIG. 6 illustrates a display showing activities used to stimulate a user's right hemisphere according to one embodiment.

FIG. 7 illustrates how the brain processes visual stimuli.

FIG. 8 illustrates how colors can be used to stimulate different brain hemispheres.

FIG. 9 illustrates how blocking half of each side of the eye and using a specific color can stimulate a specific hemisphere of the brain.

FIG. 10 illustrates Bluetooth-enabled glasses with red/blue lenses according to one embodiment of the disclosed system.

FIG. 11 is a schematic block diagram depicting an example computing system used in accordance with one embodiment of the present invention.

FIG. 12 illustrates a computing system networked to interactive exercise software, a first gaming platform, a motion-sensing device, and a second gaming platform according to one embodiment of the present invention.

FIG. 13 is a diagram depicting the interaction between the motion sensing device, computing system, display, interactive exercise software, and user according to one embodiment of the present invention.

FIG. 14 is a flowchart illustrating a method of use of the disclosed system according to one embodiment of the present invention.

FIG. 15 illustrates a user touching a hand to an opposite knee.

FIG. 16 illustrates a user touching a finger to a nose.

FIG. 17 illustrates an output of the disclosed system for a user attempting to trace a virtual circle with the user's finger and/or foot.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

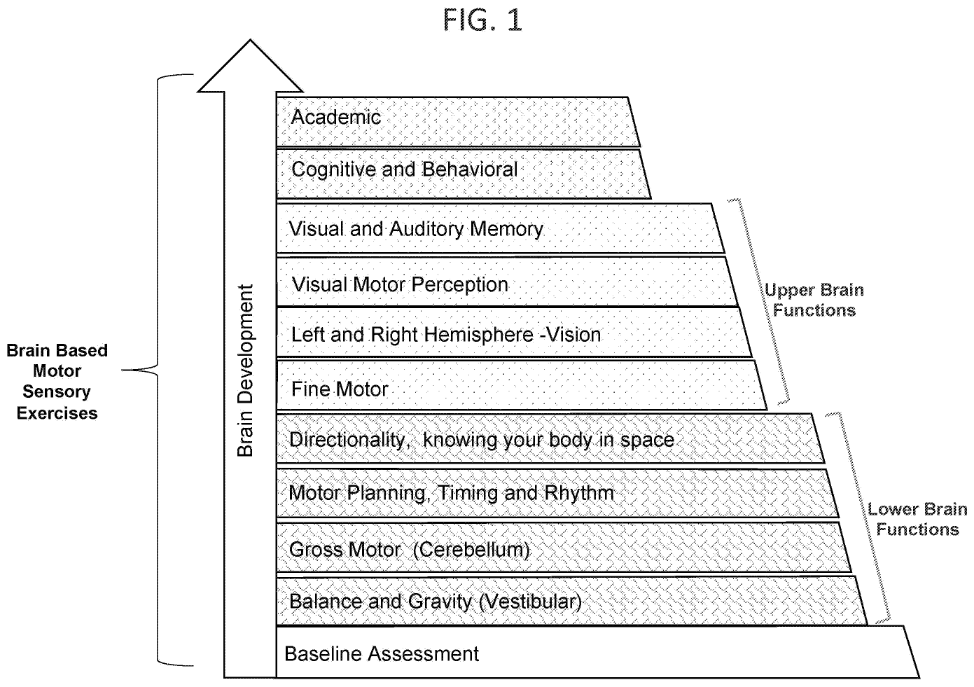

The disclosed invention, in one embodiment, is designed to activate the brain through interaction with the interactive exercise software. Therefore, as mentioned above, the disclosed system can include devices and methods that provide improved and automated therapeutic exercises for an individual; where these exercises provide physical targeted activation and cognitive skills for learning fluency in users, such as children and adults. Where physical therapy can be defined as therapy used to preserve, enhance, or restore movement and physical function impaired by disease, injury, or disability and that utilizes therapeutic exercise and assistive devices. More specifically, the system can include an exercise therapy video game and monitor system for physical targeted activation and cognitive skills for learning fluency of a user. The system can include interactive exercise software 1204 designed to provide visual movement instructions to the user via a first gaming platform 1202 having a screen 1212; a motion sensing device 1206 configured to track the body position and movements of the user and create and output raw data corresponding to the user's body positions and movements when the user is replicating the movements of the visual movements instructions; and a computing system 1208 networked to the interactive exercise software 1204, the computing system being configured to input the raw data and evaluate the accuracy of the user's body positions and movements, and provide instant feedback to the user by causing a comparison of the user's body positions and movements to the visual movement instructions that can be displayed on the screen, wherein the feedback can improve the body positions and movements of the user. The system may be capable of effecting treatment for user with cognitive development conditions through the methods employed with its exercise therapy video game. The present invention may be able to identify functional gaps in brain and body development. Because brain function can drive development, assessment of a user's functional abilities across key stages of brain and body development can be critical to the use of the present invention. Functional development is usually sequential from birth to age 12; starting with the lower brain at the bottom of the pyramid illustrated in FIG. 1. These methods may be effective at assisting users who have cerebellar dysfunction such as a learning disability, including ADD/HD, Autism, and Dyslexia.

Sensory processing refers to the way the brain receives information about surroundings from the senses and organizes it into body reactions. Measuring sensory functionality in relation to body balance and motion, i.e. the vestibular system, and body position/orientation and awareness, i.e. the proprioception system can be important because weaknesses in these senses impact self-regulation and controlled movement as well as information that is organized and sent to the upper brain for thinking skills. Exercises that can activate a user's brain and improve their neuroplasticity, may be made by targeting a user's weaknesses in maintaining their balance, sensing gravity, gross/fine motor skills, rhythm and timing, visual motor perception, and memory.

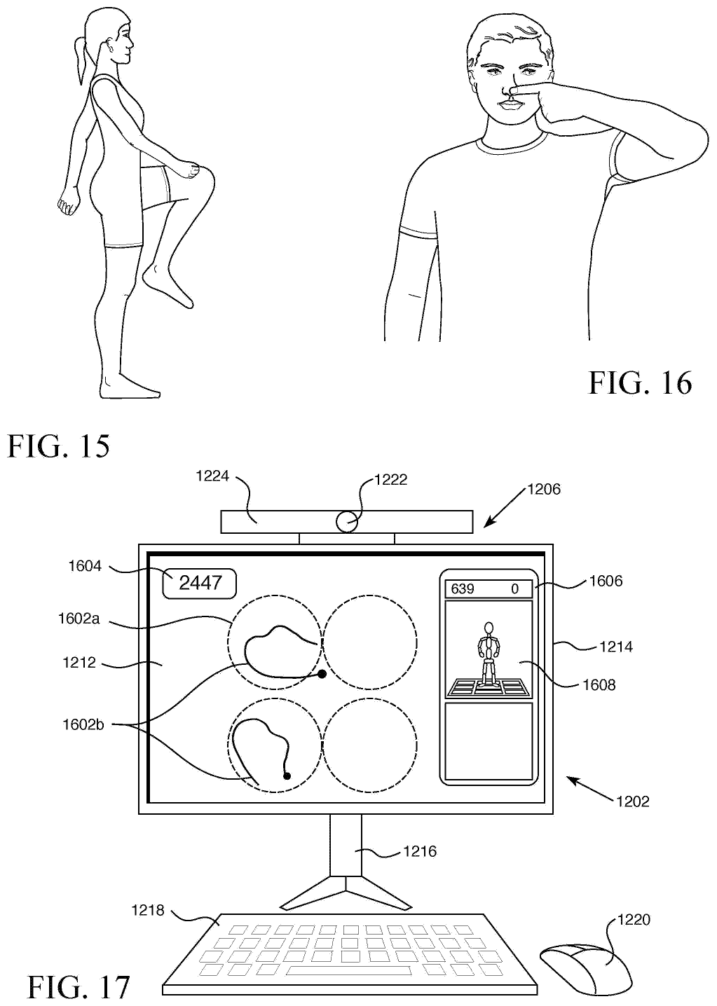

In some embodiments, the visual movement instructions can instruct the user to put a first body part of the user in contact with a second body part of the user (for example, a hand in contact with an opposite knee, as illustrated in FIG. 15, or a finger in contact with a nose, as illustrated in FIG. 16); the motion sensing device 1206 can track and output raw data for at least the position and movement of the first body part, the second body part, a third body part, and a fourth body part as well as the timing of those positions and movements; the computing system 1208 can, through the interactive exercise software, input the raw data and identify and calculate velocity and acceleration/deceleration of the first, second, third, and fourth body parts based on the raw data; and the computing system can accurately conclude if and when the first body part is in contact with the second body part. For example, this implementation of instructions may be able to provide improvement in cognitive functions for a user that is lacking synchronization with the left and right hemispheres of their brain due to lower brain disruptions. Each side of a user's brain may be able to process a specific set of functions and controls for the opposite side of the body. Both sides of a user's body should have equal strength to work together in a synchronized fashion to perform holistic skills. When a user has lower motor disruptions in the cerebellum that impact the left and right hemispheres, their brain's function and skill performance can be impeded.

More specifically, the computing system 1208 can conclude when the first body part is in contact with the second body part by determining that: the first body part and the second body part appear to be in the same position in space based on the raw position and timing data from the motion sensing device 1206, the third body part and the fourth body part have ceased moving toward each other, the first body part and the second body part have ceased moving, and the ceasing of movement by the first, second, third, and fourth body parts occurred at approximately a same point in time.

In some embodiments, the first body part can be a hand of the user, and the second body part can be a knee of the user on the opposite side of the body as the hand. For example, the hand can be a right hand, and the knee can be a left knee. Alternatively, the hand can be a left hand, and the knee can be a right knee. In further embodiments, the third body part can be an elbow of the user on the same side as the hand, and the fourth body part can be an ankle of the user on the same side as the knee. For example, if the first body part is a right hand, then the third body part can be a right elbow, and if the second body part is a left knee, then the fourth body part can be a left ankle.

In other embodiments, the first body part can be a finger of the user, and the second body part can be a nose of the user. Other body parts and combinations can be used as well, and the first and second body parts can be on the same side of the body. For example, the first and second body parts can be a right elbow and a right knee or a right hand and a left shoulder. Further, the third and fourth body parts can mirror the first and second body parts. For example, the first and second body parts can be a right hand and left knee, and the third and fourth body parts can be a left hand and a right knee. Alternatively, the first and second body parts can be a right hand and a right knee, and the third and fourth body parts can be a left hand and a left knee. Other combinations are envisioned, and the above examples are not intended to limit the combinations of contacting body parts that the computing system 1208 can detect.

In some embodiments, the visual movement instructions can have a desired user-executed outcome and a maximum score available, and the desired user-executed outcome can be when the first body part is in contact with the second body part. However, there may be cases when the desired user-executed outcome is not met by the user and, therefore, the computing system 1208 is configured to detect and determine the accuracy of the user's body positions and movements by comparing the desired user-executed outcome to the user's body positions and movements. For example, if the first body part and the second body part do not appear in the same position in space, then the computing system 1208 can conclude that the first body part is not in contact with the second body part. Further, in that case, the computing system 1208 can calculate and assign an accuracy score to the user's body positions and movements and cause the accuracy score to be displayed on screen 1212.

To calculate the accuracy score, the computing system 1208 can analyze the raw data to determine an executed outcome for the user's body movement after a predetermined amount of time, wherein the executed outcome is the position of the first body part and the second body part when the first body part was closest to the second body part in space. The computing system 1208 can then calculate the distance between the first body part and the second body part when they were closest to each other, and the distance between the first body part and the second body part when they were closest to each other can be associated with a predetermined accuracy score. If the computing system 1208 determines that the first body part has made contact with the second body part, the accuracy score can be 100. Whereas if the computing system 1208 determines that the first body part has not made contact with the second body part, the accuracy score can be less than 100. For example, if the user's hand is x distance from the user's opposite knee, the computing system can determine distance x, and the accuracy score can be 100-x.

In some embodiments, the disclosed system can further include a second gaming platform 1210 networked to the interactive exercise software 1204 and the computing system 1208. The second gaming platform 1210 can be a foot pressure sensitive device having a plurality of sensors 1226 that track foot positions and movements of the user and create raw position and timing data. Similar to the first gaming platform 1202, the second gaming platform 1210 can also output raw data to the computing system 1208. The computing system 1208 can then combine the raw data from the motion sensing device 1206 and the raw data from the second gaming platform 1210 to create a combined raw data set. This raw data set can be used by the computing device 1208 to evaluate the accuracy of the user's body and foot positions and movements and provide feedback to the user by displaying a comparison of the user's body movements to the visual movement instructions on the screen 1212.

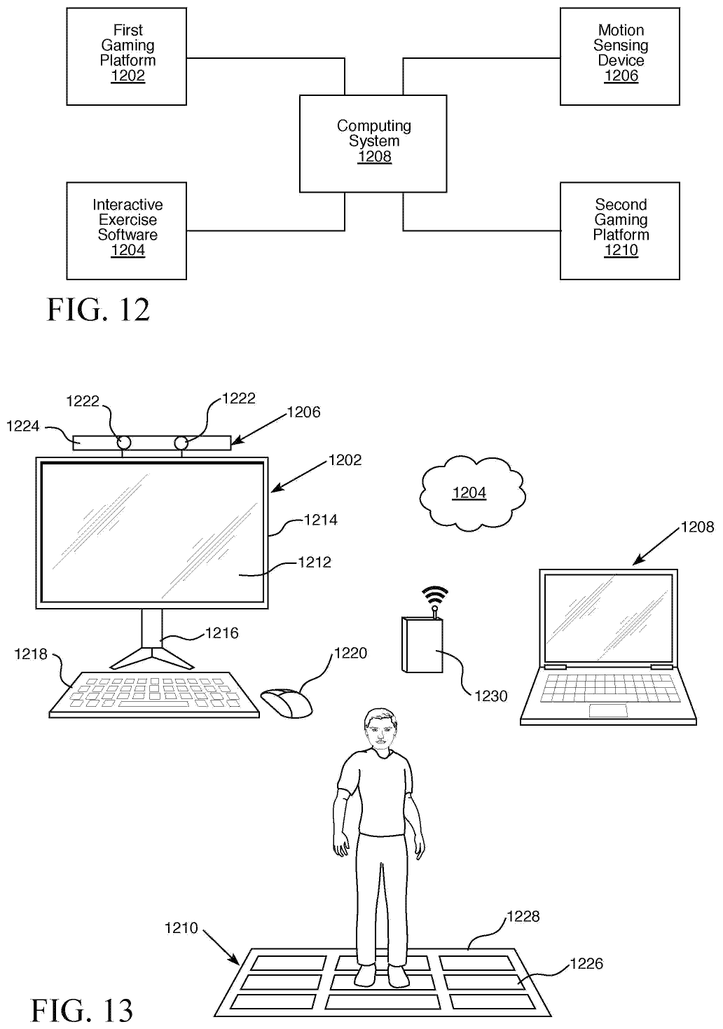

As illustrated in FIG. 12, the disclosed system can include a first gaming platform 1202, interactive exercise software 1204, a motion sensing device 1206, a computing system 1208, and a second gaming platform 1210. The computing system 1208 can be configured to network with the first gaming platform 1202, the interactive exercise software 1204, the motion sensing device 1206, and the second gaming platform 1210. As illustrated in FIG. 13, the first gaming platform 1202 may be comprised of a screen 1212, housing 1214, stand 1216, keyboard 1218, and mouse 1220; the motion sensing device 1206 may include one or more cameras 1222 housed in a housing 1224; and the second gaming platform 1210 may be comprised of sensors 1226 housed in a mat 1228.

In some instances, communication between the components may occur using a network or wireless internet connection 1230. Further, communication between the components may be unilateral (for example, the motion sensing device 1206 outputs to the computing system 1208 but the computing system does not output to the motion sensing device) and in other instances, communication may be bilateral (for example, the computing system and the first gaming platform may output and input data between each other). As illustrated in FIG. 12, the first gaming platform 1202 may output data to the computing system 1208 and input data from the interactive exercise software 1204, the interactive software may output data to the first gaming platform and input data from the computing system 1208, the motion sensing device 1206 may output data to the computing system, the computing system may output data to the interactive exercise software and input data from all other components, and the second gaming platform 1210 may output data to the computing system.

In some embodiments, some of the above-described components may be configured to function together in a single device instead of in separate devices. For example, the first gaming platform 1202, the interactive exercise software 1204, and the computing system 1208 may all be included in one collaborative device. Therefore, the motion sensing system 1206 and the second gaming platform 1210 may network directly with the collaborative device instead of outputting directly to, for example, the interactive exercise software 1204 or the computing system 1208.

In some embodiments, the first gaming platform 1202 can have a screen 1212 to display images and movement instructions provided by interactive exercise software 1204. Further, the screen 1212 of the first gaming platform 1202 can be a touch screen that enables a user to make selections provided by the interactive exercise software 1204. In other embodiments, a keyboard 1218 or mouse 1220 can be paired with the first gaming platform 1202, so that the user can make selections on the screen 1212.

In some embodiments, as mentioned above, the interactive exercise software 1204 works with the first gaming platform 1202 to create an exercise therapy video game for a user. In combination with the motion sensing device 1206, the overall system can provide an exercise therapy video game with a monitoring system that provides physically targeted activation and cognitive skills for learning fluency to stimulate the user. The interactive exercise software 1204 can, through the first gaming platform 1202, present the user with therapeutic exercises or games that request the user to complete specific body positions and movements. The computing system 1208 described above can then take raw data about the user's body position and movements from the motion sensing device 1206 and manipulate it to determine whether the user has completed the therapeutic exercises or games.

In some cases, the interactive exercise software 1204 may be stored in the first gaming platform 1202 with or without the computing system 1208. In cases where the interactive exercise software 1204 and computing system 1208 are combined with the first gaming platform 1202, a network or wireless internet connection 1230 may not be required to communicate information and data between components. Further, in some embodiments, the second gaming platform 1210 is not a required element for the exercise motion system to function and, therefore, is not part of the overall system.

For example, if a second gaming platform 1210 is not used, the only remaining component required for communication purposes may be the motion sensing device 1206 which, due to its close location near the first gaming platform 1202, could be attached directly via a cord or cable (ex: USB cable, HDMI cable, or any other standard connection cable). If a second gaming platform 1210 is used, it could also be attached to the first gaming platform 1202 via a cord or cable, therefore eliminating any need for a network or wireless internet connection 1230.

In some embodiments, the motion sensing device 1206 can include, but is not limited to, one or more cameras 1222 housed in a housing 1224, as illustrated in FIG. 13, that tracks specific joints and/or body position and movements of the user as well as the time it takes a user to move into desired body positions and movements. The camera(s) 1222 can take several measurements per second of the body positions and can transfer its raw position and timing data to the computing system 1208. As illustrated in FIG. 13, the motion sensing device 1206 can be positioned above the screen 1212 of the first gaming platform 1202 to obtain the most accurate body position and movement data. More specifically, since the movement instructions may be displayed on the screen 1212, having the motion sensing device 1206 as close to the screen as possible enables the motion sensing device to easily and accurately track body positions and movements of the user.

In some embodiments, the second gaming platform 1210 can be comprised of sensors 1226 located on or under, or housed within, a mat 1228, such as a dance mat, for tracking foot positions and movements, as well as timing of those foot positions and movements, of a user. Therefore, as the user moves around on the mat 1228, the sensors 1226 are triggered by pressure from the user's feet and produce raw position and timing data points that then get sent to the computing system 1208, as described above.

As mentioned above, the computing system 1208 can be a stand-alone device, as illustrated in FIG. 12, or it can be one component of an inclusive device that includes one or more of the first gaming platform 1202, interactive exercise software 1204, motion sensing device 1206, and second gaming platform 1210. The computing system 1208 can be configured to accept raw data from the motion sensing device 1206 and, as relevant, from the sensors 1226 of the second gaming platform 1210. It can then analyze the raw data to determine what types of movements the user is making. Once the user's specific movements have been determined, the computing system 1208 can compare the user's movements to the movement instructions to determine if the user is properly completing the therapeutic exercises or games.

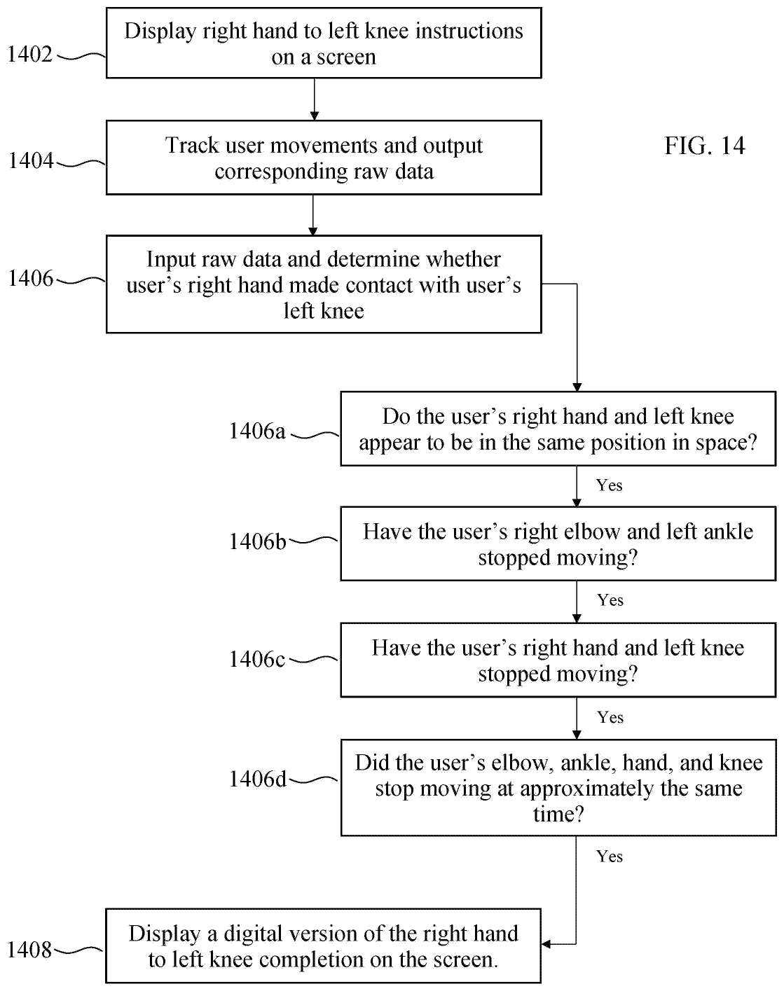

More specifically, the computing system 1208 can input the measurements of the user's body position and movements (i.e., the raw data from the motion sensing device 1206 and second gaming platform 1210) and cross-reference and compare a plurality of the measurements against each other to accurately and precisely determine positions of the user's joints and body parts. For example, as illustrated in FIG. 14, if the movement instructions inform the user to touch the user's hand to opposite knee 1402, the user may attempt to follow the instructions, as illustrated in FIG. 15. As the user moves, the motion sensing device 1206 can track spatial positions of the user's body parts as well as timing of the positions, and the sensors 1226 of the second gaming platform 1210 can, if used, sense foot placement (i.e., if a foot is on or off the ground and, if on the ground, its position on the mat 1228) and timing of placement. The motion sensing device and second gaming platform can then output the raw position and timing data 1404. The computing system 1208 can then input these raw position and timing data and can use the data to determine if a threshold of specific positions and movements has been met 1406.

This threshold can, for example, include if/then protocols such as “if the following criteria have been met, then the conclusion is that the user's hand is touching the user's opposite knee.” In some embodiments, the criteria can include, but are not limited to, questions such as: (1) Do the user's hand and opposite knee near appear to be in the same position in space 1406a (i.e., can the motion sensing device 1206 no longer distinguish between the two body parts and/or do the sensors 1226 only sense one foot on the mat 1228)? (2) Have the user's elbow and opposite ankle stopped moving 1406b (i.e., have these two body parts accelerated and decelerated, and do they now have a velocity near or equal to zero)? (3) Have the user's hand, and opposite knee stopped moving 1406c (i.e., have these two body parts accelerated and decelerated, and do they now have a velocity near or equal to zero)? (4) Did the user's elbow, ankle, hand, and knee stop moving at approximately the same time 1406d (i.e., did each pairing of body parts reach zero, or near zero, velocity at approximately the same time)?

If the threshold is met (i.e., the answer to all of the above questions is “yes”), then the computing system 1208 can output information to the interactive exercise software 1204 confirming that the threshold has been met, and the interactive exercise software can then send instructions to the screen 1212 of the first gaming platform 1202 to display a digital version of the hand making contact with the opposite knee 1408. This visual provides automatic feedback to the user that the movement instructions have been fully completed by the user.

As a user is provided movement instructions by the interactive exercise software 1204, the user can be assigned a score less than or equal to a maximum score, wherein the assigned accuracy score 1604 indicates how accurately the user replicated the provided instructions, as briefly mentioned above. A maximum score can equate to the user replicating the movement instructions perfectly or near-perfectly. For example, if the movement instructions are to use a finger and/or foot to trace a traceable circle or sideways figure eight track 1602a that is shown on a screen, as illustrated in FIG. 17, and the user traces the circle near-perfectly resulting in a user-created line 1602b, the user can be assigned a maximum score. Any variation off of the circle resulting in a user-created line 1602b (i.e., moving a finger or foot off the traceable line), as illustrated in FIG. 17, can decrease the user's score according to the extremity of the variation or mistake.

More specifically, if the user's finger is x distance away from the circle, the user's score will decrease less than if the user's finger is x+1 inch away. In another example, if the user's hand is x distance from the user's opposite knee, the user's score will decrease less than if the user's hand is x+1 inches away. A virtual avatar 1608 can also be displayed on the screen 1212 in a feedback box 1606, and the avatar can mirror the user's body positions and movements.

In some embodiments, movement instructions can be provided for a plurality of different physical activities, and each activity can have incrementally difficult levels for each physical activity. Therefore, for example, in order for a user to be provided movement instructions for a level two movement, the user may need to score at or above a threshold or “optimal” score in level one. This optimal score is lower than the level one maximum score but indicates the user has been able to meet or surpass a predetermined accuracy threshold for the level one physical activity.

In some embodiments, the disclosed invention is designed and configured to use a compared weighting system to compare left hemisphere-based physical activities to right hemisphere-based physical activities and to measure cerebellum or hind brain to higher level cortex activities. The system can then produce a solution, such as one or more series of interactive physical therapy exercises, that is specific to the user and that targets the physical activities at which the user performed worst. Under the disclosed system, training activities can modify as the user continues to complete them, ensuring continuous physical targeted activation and cognitive skills improvement. Therefore, as the user adapts to the activity, the system can modify the activity by increasing the difficulty of the physical targeted activation.

The various cognitive brain functions impacted by physical targeted activation, as illustrated in the pyramid of FIG. 1, can be included in assessment and training of the user and can include, but are not limited to, vestibular, cerebellum, motor planning and timing, directionality, fine motor, visual motor perception, and visual and auditory memory. After determining any imbalances or weaknesses, the disclosed system can correct those imbalances or weaknesses through training exercises and integrate those corrected imbalances or weaknesses into later exercises or daily routines. The system is capable of taking an assessment or evaluation at a first point in time and then measuring gaps and improvements between that assessment and a user's exercise to determine if the user is, in fact, improving and, if so, it can determine future exercises for the user. The assessments discover brain body functional gaps and then the training sessions will target either a left hemisphere or right hemisphere targeted program.

Several training activities can be used to improve the cognitive and physical health of an individual. The training exercises can target and strengthen specific hemispheres of the brain through incorporation of side-specific physical activities focused on balance, motor, timing and rhythm, directionality, fine motor skills, vision, auditory, and memory. For example, if the user is worse at one activity on their left side than their right side, the training exercises can focus on left-side activities and, through continued repetition of the left-side activities, strengthen neural connections in the opposite (i.e., right) hemisphere.

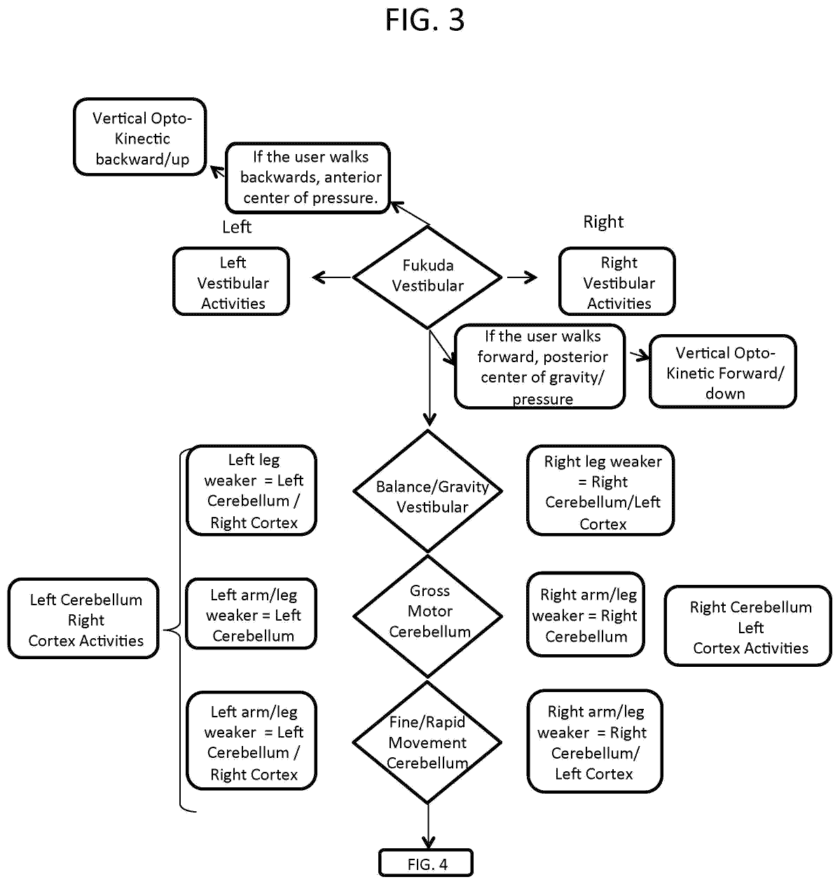

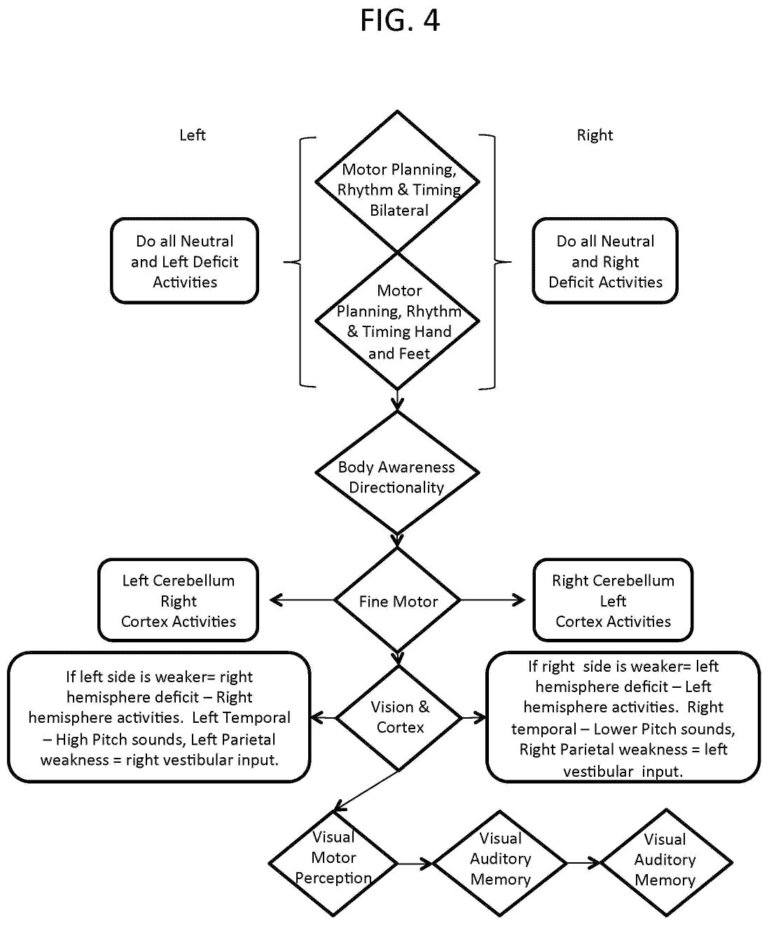

In one embodiment, as illustrated in FIGS. 3 and 4, the disclosed system can expose the user to a plurality of exercises to assess a user's baseline capabilities and determine if and where a user needs improvement. For example, as illustrated in FIG. 1, the user's vestibular brain functions can be initially assessed by testing balance and gravity, then the user can next be assessed for gross motor and cerebellum function imbalances or weaknesses by testing large muscles, after which the user can be assessed for motor planning, timing and rhythm, directionality (i.e., left/right body awareness), fine motor, left and right hemisphere vision, visual motor perception, and visual and auditory memory.

More specifically, the user may initially have a baseline assessment completed that first determines the user's heart rate and completes a face scan and then assesses brain function. More specifically, the user's vestibular brain functions can be assessed by, for example, implementing the Fukuda Stepping Test for Vestibular Function and/or a balance test. The user gross motor and cerebellum assessment can use a finger to nose test, as illustrated in FIG. 16, a figure eight's test, as illustrated in FIG. 17 and described above, and/or a pancake hands test. The motor planning, timing, and rhythm assessment can use, for example, a cross crawl test and/or a dance sequence test. The remaining assessments all have additional tests that can be completed for a proper assessment of a user's brain functionality.

The system can then deliver a series of activities across all the functional areas targeted for improvement. For example, if the system determines that a user has a balance and gravity (vestibular) weakness, the system can deliver a series of therapeutic training exercises or a series of activities such as hopscotch and/or balance/yoga.

In an alternative embodiment, the disclosed system can test the user's vestibular brain functions, and, if the user's performance meets a predetermined threshold, the system can test the user's gross motor and cerebellum functions. It can continue working up the pyramid to the test each of the functions until it determines if and where a user needs improvement. Then, when the system determines an area that needs improvement, the assessment portion of the disclosed system can be paused and the training portion can commence. Therefore, in this embodiment, instead of delivering a series of activities across all the functional areas targeted for improvement, the system can target activities for a single weak area until that area improves, and then move on to a new weak area.

In one embodiment, the system may provide a person with all of the available therapeutic exercises. The user can start at level one for all of these physical exercises, train at level one for all exercises, and, as the user improves in each activity, can then move on to the next level for the corresponding exercises. In one embodiment, each exercise has three levels of difficulty. Additionally, as the user's physical abilities improve through use of specific therapeutic exercises, those therapeutic exercises can be integrated into new therapeutic exercises that focus on other physical capabilities in order to improve overall performance. For example, if an individual has tested poorly in one therapeutic exercise, the system can present the individual with that physical exercise while simultaneously incorporating skills such as motor planning and visual tracking in order to achieve a better, longer lasting change. Additionally, the system can use color and sound to amplify the effectiveness of the activity.

In another embodiment, the system can start a user with a specific set of therapeutic exercises at level one (for example, level one for all activities that focus on balance and gravity). The user can move up through additional levels of those exercises until each of those levels are completed. Once the user has completed all of the levels of all of the exercises associated with a specific set (for example, balance and gravity), the system can test the user on the next set of therapeutic exercises (for example, gross motor exercises associated with large muscles). If the assessment determines that the user requires training within this next set of exercises, the user can once again start at level one for all relevant exercises (for example, gross motor exercises associated with large muscles) and work his or her way up through the levels until all levels of all relevant exercises are completed. Once all levels of all relevant exercises are completed, the user can be tested, and possibly trained, on the next set of therapeutic activities.

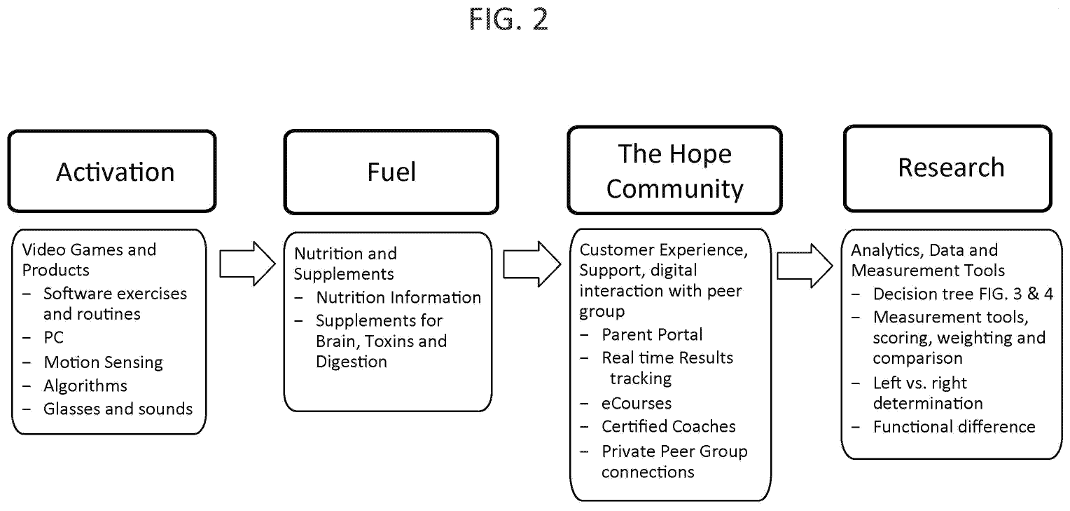

In some embodiments, as illustrated in FIG. 2, the disclosed invention can include Bluetooth-enabled glasses, a gaming platform such as a dance mat, one or more cameras, a computing system, a motion sensing device, and a system and method that includes an assessment of physical capabilities, therapeutic exercise training activities, data collection methods, data analysis methods, communication capabilities, internet capabilities, and an online community platform that can engage and offer support to users and provide nutritional information.

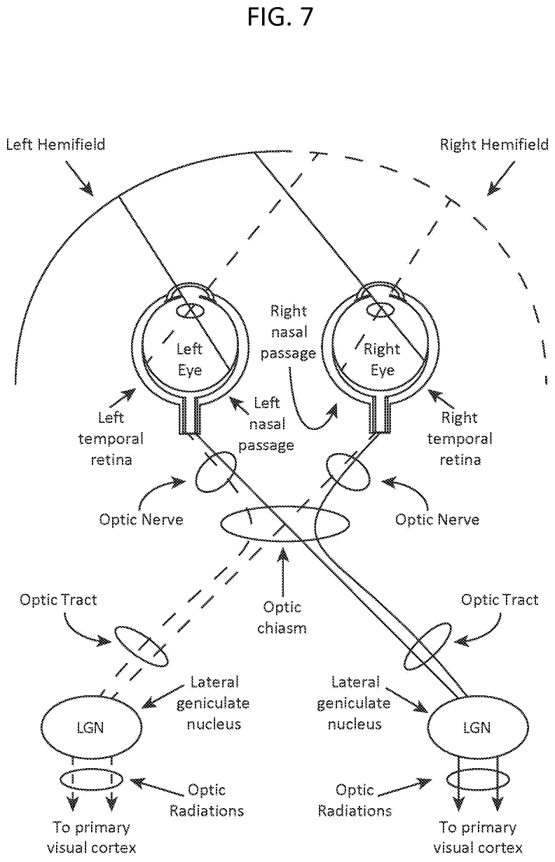

In general, visual input from either eye can send a signal to one hemisphere of the brain depending on which direction the visual input is coming from. For example, as illustrated in FIG. 7, visual input from a person's right side is directed to the left hemisphere of the brain and visual input from a person's left side is directed to the right hemisphere of the brain. Additionally, visual input from the top part of a person's vision stimulates the temporal lobe and visual input from the bottom part of a person's vision stimulates the parietal lobe. Therefore, on-screen elements displayed to the user can be used during the therapeutic exercises to stimulate different hemispheres of the user's brain based on where the on-screen elements are located on the screen (ex: right, left, top, or bottom).

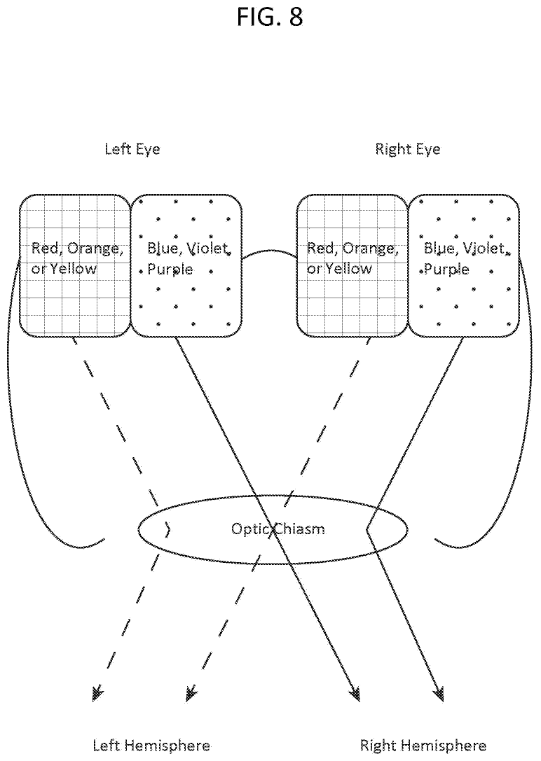

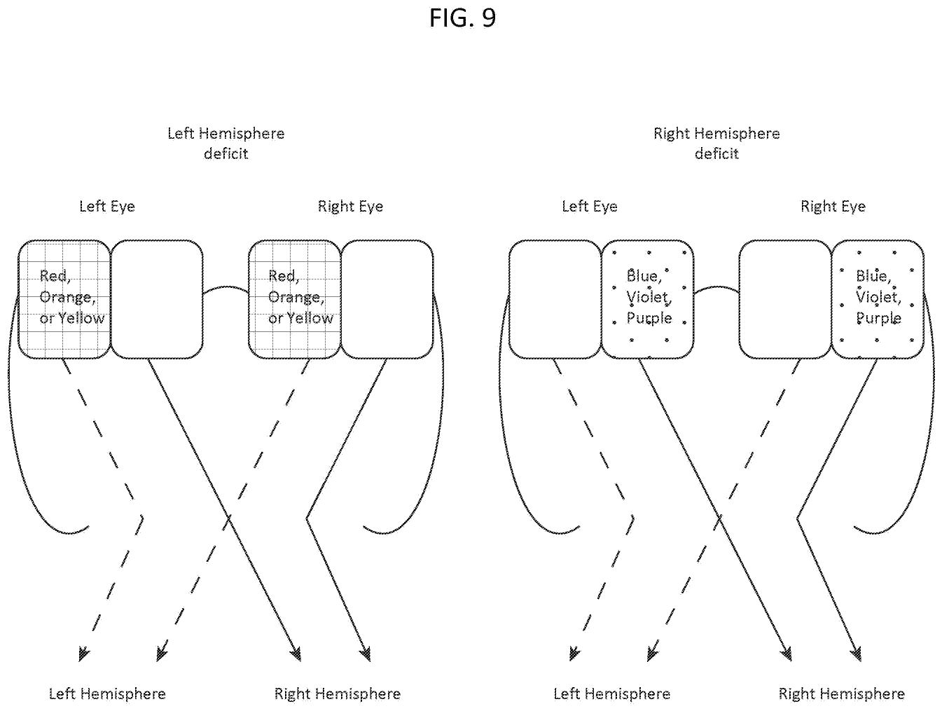

Additionally, color and sound can stimulate the brain. For example, red can stimulate the left hemisphere of the brain, while blue can stimulate the right hemisphere of the brain, as illustrated in FIGS. 8 and 9. Further, the left hemisphere of the brain can be stimulated by high tones, while the right hemisphere of the brain can be stimulated by low tones.

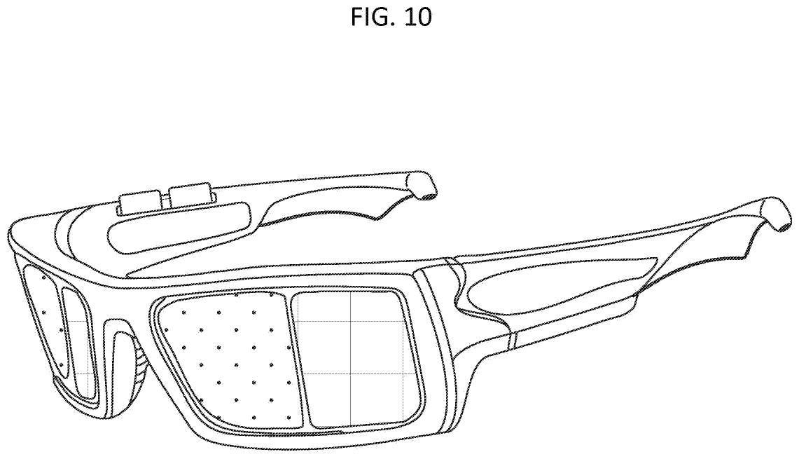

The Bluetooth-enabled glasses, as illustrated in FIG. 10, can be glasses with red/blue lenses. In one embodiment, the glasses can be standard-size safety glasses with colored lenses. As illustrated in FIGS. 8 and 10, the left half of both lenses can be red, and the right half of both lenses can be blue. In another embodiment, as illustrated in FIG. 9, one half of each lens can be blocked or black. For example, the left half of each lens may be red while the right half of each lens may be black. In another example, the right half of each lens may be blue while the left half of each lens may be black. In another embodiment, the lenses are easily removable from the glasses so that other lenses, such as clear or tinted lenses, can be replaced in the glasses. In a further embodiment, the color of the lenses can automatically change based on feedback provided by the interactive exercise software. For example, if the game communicates to the lenses that they should turn blue, the lenses can then turn blue.

Further, as illustrated in FIG. 10, the Bluetooth-enabled glasses can have earbuds built into the ends of the glasses in order to coordinate music play with the visual movement instructions and, therefore, the user's body positions and movements, although the music play can be played using any audio system having speakers. The glasses can connect to any Bluetooth-enabled device. Therefore, the wearer can have simultaneous visual and auditory input when wearing the Bluetooth-enabled glasses. For example, the glasses or other speaker may play detailed, high frequency tones or music with lyrics and may primarily use the notes G, B, and A. Alternatively, the glasses or other speaker may play low frequency music with no lyrics and may primarily use the notes C, D, E, and F. In order to play sound, in some embodiments, the glasses or other speaker may require a battery, such as at least one rechargeable or disposable battery.

In some embodiments, the auditory feature can be provided through the computing device or through a separate speaker. Regardless of how the sound reaches the user, the system can control the sound that is played to the user so that the proper frequency and notes correspond with specific therapeutic exercises.

In addition to using the glasses for the therapeutic exercises, the system can include vision-specific therapeutic activities. These vision-specific therapeutic activities can be used on their own, or they can be integrated with other therapeutic exercises for a more effective result in those exercises. For example, the system can train balance by having the user complete a physical activity, or it can train balance by combining a physical activity with visual or auditory input (for example, the glasses).

In one embodiment, one or more of the therapeutic exercises used in the system involve use of a person's whole body, which can activate the user's brain across specific left and right hemispheric regions and can increase learning. These whole-body therapeutic exercises or training exercises can be hands-free and integrate motion, motor-sensory, rhythm and vision through the use of the Bluetooth-glasses, motion sensing device, and the system and method described herein. Some of the activities can incorporate visual/picture reading, spelling and math, additional sensory-motor, or hand-eye fine motor activities.

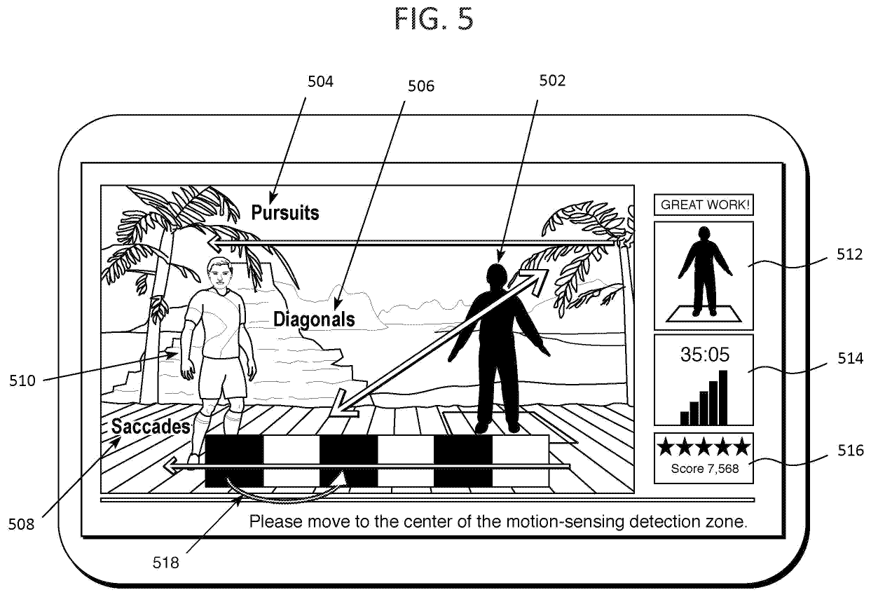

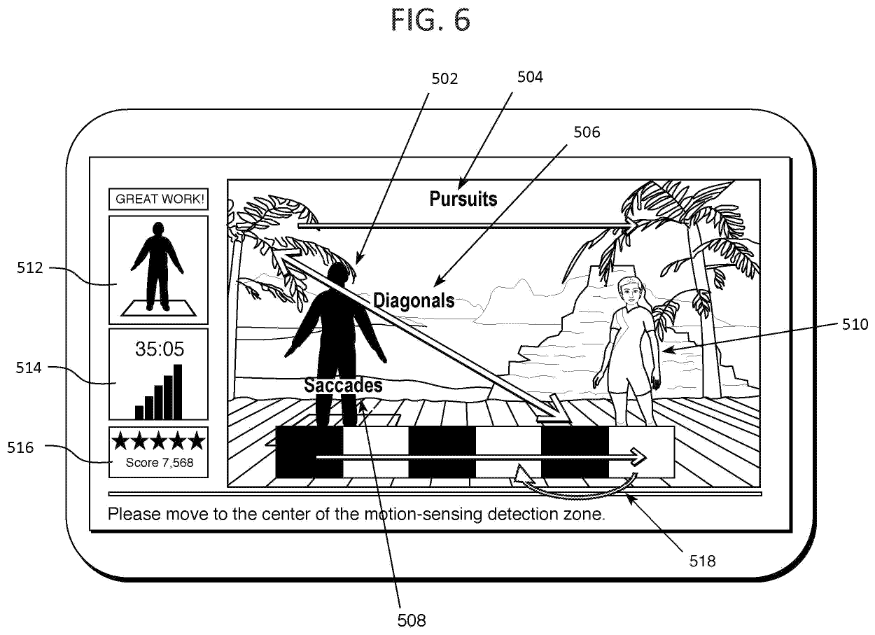

For example, as illustrated in FIGS. 5 and 6, one or more therapeutic exercises or activities can be displayed to a user on a screen. More specifically, as illustrated in FIG. 5, a user can be presented with right-hand specific and right-leg specific activities using an on-screen avatar 502 that can be located on, for example, the right side of the screen. More specifically, objects displayed on the screen, such as horizontal pursuits 504, can move slowly from right to left, objects such as diagonal pursuits 506 can move in a diagonal pattern from the bottom left to the upper right portion of the screen, objects on the screen, such as trains, logs, or waves, otherwise known as saccades 508 or fast-moving objects, can move quickly move from right to left, causing a user's eyes to jump from left to right as illustrated by arrow 518, popups displayed may appear on the right side of the screen, and an activity that involves drawing or tracing objects may occur on the right side of the screen. Additionally, the user may listen to high frequency sounds or music during the activity, and the screen and on-screen elements may be primarily warm colors such as, but not limited to, red, yellow, and orange. To help the user, the screen may show an on-screen instructor 510 that is used to illustrate visual movement instructions to the user. Off to the side, as illustrated in FIG. 5, the display may show a second avatar 512 that is removed from the environment, a timer 514 to show time elapsed while completing the activity, and a score 516 so that the user knows how well he or she is accomplishing the activity.

In some embodiments, a user can be presented with left-hand specific and left-leg specific activities using an on-screen avatar 502 that can be located, for example, on the left side of the screen, as illustrated in FIG. 6. More specifically, the objects displayed, such as horizontal pursuits 504, can move slowly from left to right, objects such as diagonal pursuits 506 can move in a diagonal pattern from the bottom right to the upper left portions of the screen, objects on the screen, such as trains, logs, or waves, otherwise known as saccades 508 or fast-moving objects, can move quickly from left to right, causing a user's eyes to jump from right to left as illustrated by arrow 518, popups displayed may appear on the left side of the screen, and an activity that involves drawing or tracing objects may occur on the left side of the screen. Further, the user may listen to low frequency sounds or music during the activity, and the screen and on-screen elements may be primarily cool colors such as, but not limited to, green, blue, navy, and purple. As with the right-sided activities described above, the system may assist the user by showing an on-screen instructor 510 that is used to illustrate desired movements to the user. Off to the side, as illustrated in FIG. 5, the display may show a second avatar 512 that is removed from the environment, a timer 514 to show time elapsed while completing the activity, and a score 516 so that the user knows how well he or she is accomplishing the activity.

Because the activity on screen can coordinate with the sound being played, a user can have an integrated learning experience. For example, the activity on screen may have the user targeting left hand movements and left leg movements, the user may be looking through the Bluetooth-enabled glasses that have a partially blue lens, and the system may be playing low frequency music with no lyrics. However, while the auditory element can be integrated with another function such as vision (through the glasses) or motor planning (through physical activity), the auditory element has the option of being isolated and used as a solo training element.

In one embodiment, the motion sensing device 1206 used in coordination with the training activities can be comprised of one or more cameras 1222 in a housing 1224, smart clothing that is motion trackable, or any other device that can track balance, head and body positions, 2D and 3D motions, and that can record data analytics to a back-end server.

The motion sensing device 1206 can have precision tracking tools that track fine motor activities such as, but not limited to, finger movements, nose movements, feet movement, etc. Additionally, it can track gross motor activities, visual motor activities, balance, and gravity to determine where the user is in space. When tracking gravity, the interactive exercise software 1204 can determine and use the user's body's center of mass to further determine how the user rotates in gravity and space.

In some embodiments, if the user is using a gaming console, the gaming console can network with a motion sensing device 1206 that communicates with a handheld controller that may be embedded with custom firmware that controls the system operation. For example, the handheld controller, such as a Wii Remote® for Nintendo Wii® or the Move Motion Controller for PlayStation®, can sense an infrared light beam sent from the gaming console and can communicate back its position relative to the console using Bluetooth® technology. Alternatively, the motion sensing device 1206 may not communicate with a handheld controller but may be a motion sensor that can pick up on the user's movements using one or more cameras 1222 (for example, infrared laser light cameras). For example, the motion sensing device may be an Orbbec® 3D device, a VicoVR® sensor device connected to Android® and iOS smart devices or other gaming platforms, a Kinect® for personal computer (hereinafter “PC”) or for the Xbox®, a Leap® Motion Controller connected to an Apple® or Microsoft® device, or a mobile phone using a camera as a motion sensor. In another embodiment, a holographic computer, such as the Microsoft® HoloLens® or a virtual reality headset, such as the Oculus Rift®, can be used to track a user's body position and movements in combination with a therapeutic exercise/training activity. The activities and moving objects could be projected onto the lens for a user and the user could move in reaction to what is displayed.

As illustrated above, the system described herein can collect and record data based on the user's body position and movements for each series of therapeutic exercises/interactive activities and can output the raw data, which can be input into the computing system. The system can further compare the user's position and movement data from each series of interactive activities, which are each weighted and scored, to a desired user-executed outcome for each of the corresponding interactive activities and can display the comparison to the user in the form of feedback, which can be immediate feedback. This comparison is used to determine if and when the user has successfully completed each series of interactive activities. Additionally, the individual games disclosed herein can be loaded onto the gaming console wirelessly or can be stored on a disk and loaded onto the gaming console through the disk reader.

The motion sensing device can be used with a computing system such as, but not limited to, a desktop computer, a laptop computer, a television, such as a smart TV, a tablet, such as, but not limited to, a Microsoft Surface®, iPad®, Samsung Galaxy®, or Google® Nexus®, and/or a mobile device such as an Android® or iOS device. In some embodiments, the therapeutic exercises can be coordinated with the motion sensing device to be implemented on, for example, a television, a personal computer, a monitor, a surface projector, or a tablet. The recorded data, in some embodiments, can be accessible to a user or other individual through a computer that is connected to the Internet.

One example of a basic, vestibular training activity that can be used in the system involves near-far focusing. When completing this activity, a user can hold a finger in front of his or her face, identify a faraway small object or have a faraway object on screen, and look back and forth at the thumb and object at a moderate pace, ideally getting a clear image each time. Another example of a basic, vestibular training activity that can be used in the system involves focusing on a target after spinning in circles. When completing this activity, the user can spin in circles a predetermined number of times (as indicated in the on-screen visual movement instructions) and then attempt to focus on an object on screen by pointing at the object. In some embodiments, the user's eyes can be open or closed while spinning, and the position of the user's finger can be reflected by an on-screen arrow. The user is, therefore, attempting to find and focus on an on-screen object/target and then attempting to hold the virtual arrow steady on the on-screen object/target by moving the arrow to the on-screen object/target and holding the user's finger steady in space.

For either of the above-described activities, the motion sensing device can collect and output raw data associated with the head position, finger position, and body movements, the computing system can input the raw data and analyze it to determine if the user's body positions and movements are out of position, and the computing system can then transmit feedback to the gaming platform screen to show the user how accurate the user's body position and movements were. Additionally, the computing system can use the raw data and the interactive exercise software to score the user based on the difference between desired body position and the executed body position.

Another example of a basic, vestibular training activity used in the system involves creating figure eights with hands and feet, as indicated in FIG. 17. This activity also trains for gross motor/cerebellum and visual improvements. When completing this activity, a user can user his or her hand and/or foot to push a virtual object, such as a dot, around a sideways figure eight track 1602a a predetermined number of times (for example, three times). The motion sensing device 1206 can track the user's physical positions and movements as well as the speed and accuracy of the user's movements and compare the position/movements to a desired user-executed outcome, which can be displayed to the user in the form of immediate feedback as a user-created line 1602b.

One example of a gross motor/cerebellum training activity used in the system involves one-foot hopping. When completing this activity, a user can stand on one foot with the other leg in the air and bent at the knee. The user can then repetitively hop side-to-side, using the same leg, so that the user's avatar hops over a virtual line on the screen a certain number of times. In one embodiment, there are different levels to the activity. For example, the first level could require the user to hop 10 times, the second level could require the user to hop 10 times with a metronome element of 60 beats per minute, and a third level could require the user to hop 10 times to an irregular beat, but in a pattern. In one embodiment, music can be played while the user is doing the activity and the screen color can be chosen based on which foot the user is hopping on. For example, the instructions provided may be for the user to hop on his or her right foot, while listening to high frequency music and viewing a screen with the colors red, yellow, and orange. During the activity, the motion sensing device 1206 can track and record accurate hops.

Another example of a gross motor/cerebellum training activity used in the system involves a user hopping on both feet, wherein the feet are either in the same or difference positions on the mat 1228 of the second gaming device 1210. When completing this activity, a user can stand on both feet and repetitively hop up and down while using on-screen movement instructions to determine the position of his or her feet. For example, movement instructions could indicate that the user should land with both feet on a first sensor and then hop to simultaneously move one foot onto a second sensor and another foot onto a third sensor. The user can continue jumping with both feet and landing on designated spots/sensors until the movement instructions indicate that the activity is complete.

One example of a higher-level, visual and auditory memory exercise used in the system involves identifying digit span. When completing this therapeutic exercise/activity, a user can, while standing, view a certain number of digits on a screen for a certain number of seconds. The numbers can then be removed from the screen and the user may be asked to repeat the numbers in order or point to the screen to indicate which numbers came first, second, third, etc. In one embodiment, there may be different levels to the activity. For example, in the first level, the user can view three digits for three seconds or four digits for four seconds. Once the user successfully completes the activities in the first level, the user can move on to the second level, wherein the user can view five digits for five seconds or six digits for six seconds. Once the user successfully completes the activities in the second level, the user can move on to the third level, wherein the user can view seven digits for seven seconds.

In one embodiment, data can be collected from the therapeutic exercises and can be analyzed and used for research. The disclosed system can also include tracking and immediate feedback to a user completing the exercises by utilizing the motion sensing device for body, hand, feet, and facial recognition.

In one embodiment, the disclosed interactive exercise software is digital and downloadable. Users can then customize plans for therapeutic exercises that can be tailored to a specific individual. The plan can be designed so that a non-user can monitor the user. The interactive exercise software can also contain an online reporting system that indicates how many minutes the user has spent doing the activity and can track the player's progression over time. In one embodiment, an online leader board may exist that can encourage competition and experience building.

The online community platform can include access to currently available activities, new activity releases, courses, webinars, a certified coaching program, and various types of support functions. The courses can include videos, written content, a posting board, controlled class comments, and connection to social media outlets. Content can be released lesson by lesson. The webinars can be enabled to accept questions prior to their release. The community platform can also have means to accept feedback.

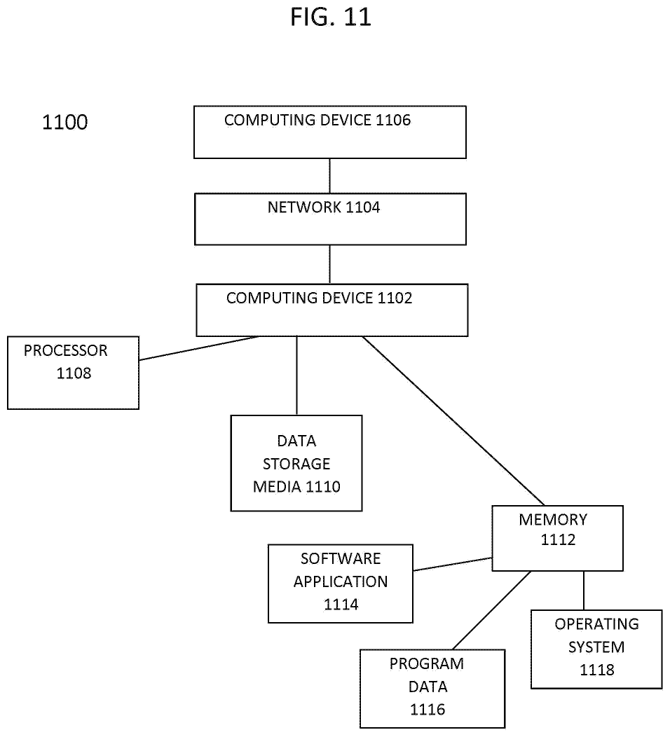

In some embodiments, the system described herein uses a computing system to carry out at least some of the various functions described herein. FIG. 11 is a schematic block diagram of an example computing system 1100. The example computing system 1100 includes at least one computing device 1102. In some embodiments the computing system 1100 further includes a communication network 1104 and one or more additional computing devices 1106 (such as a server).

The computing device 1102 can be, for example, be a computing device 1102 located in a user's home, school, or other place of business. In some embodiments, computing device 1102 is a mobile device. The computing device 1102 can be a stand-alone computing device or a networked computing device that communicates with one or more other computing devices 1106 across a network 1104. The additional computing device(s) 1106 can be, for example, located remotely from the first computing device 1102, but configured for data communication with the first computing device 1102 across a network 1104.

In some examples, the computing devices 1102 and 1106 include at least one processor or processing unit 1108 and system memory 1112. The processor 1108 is a device configured to process a set of instructions. In some embodiments, system memory 1112 may be a component of processor 1108; in other embodiments system memory 1112 is separate from the processor 1108. Depending on the exact configuration and type of computing device, the system memory 1112 may be volatile (such as RAM), non-volatile (such as ROM, flash memory, etc.) or some combination of the two. System memory 1112 typically includes an operating system 1118 suitable for controlling the operation of the computing device 1102, such as the WINDOWS® operating systems or the OS X operating system, or a server, such as Windows® SharePoint Server. The system memory 1112 may also include one or more software applications 1114 and may include program data 1116.

The computing device 1102 may have additional features or functionality. For example, the computing device 1102 may also include additional data storage devices 1110 (removable and/or non-removable) such as, for example, magnetic disks, optical disks, or tape. Computer storage media 1110 may include volatile and nonvolatile, removable and non-removable media implemented in any method or technology for storage of information, such as computer readable instructions, data structures, program modules, or other data. System memory, removable storage, and non-removable storage are all examples of computer storage media. Computer storage media 1110 includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by the computing device 1102. An example of computer storage media 1110 is non-transitory media.

In some examples, one or more of the computing devices 1102 and 1106 can be located in an establishment. In other examples, the computing device 1102 can be a personal computing device, such as a personal computer or gaming device, that is networked to allow the user to access and utilize the system disclosed herein from a remote location, such as in a user's home, office or other location. In some embodiments, the computing device 1102 is a smart phone, tablet, laptop computer, smart TV, personal digital assistant, or other mobile device. In some embodiments, system operations and functions are stored as data instructions for a smart phone application. A network 1104 facilitates communication between the computing device 1102 and one or more servers, such as an additional computing device 1106, that hosts the system. The network 1104 may be a wide variety of different types of electronic communication networks. For example, the network 1104 may be a wide-area network, such as the Internet, a local-area network, a metropolitan-area network, or another type of electronic communication network. The network 1104 may include wired and/or wireless data links (such as through 3G or 4G networks). A variety of communications protocols may be used in the network 1104 including, but not limited to, Wi-Fi, Ethernet, Transport Control Protocol (TCP), Internet Protocol (IP), Hypertext Transfer Protocol (HTTP), SOAP, remote procedure call protocols, and/or other types of communications protocols.

In some examples, the additional computing device 1106 is a Web server. In this example, the first computing device 1102 includes a Web browser that communicates with the Web server to request and retrieve data. The data is then displayed to the user, such as by using a Web browser software application. In some embodiments, the various operations, methods, and functions disclosed herein are implemented by instructions stored in memory. When the instructions are executed by the processor 1108 of the one or more computing devices 1102 or 1106, the instructions cause the processor 1108 to perform one or more of the operations or methods disclosed herein.

One example embodiment of the disclosed system includes a motion control, monitoring, and assessment system comprising a motion sensing device having precision tracking for fine motor activities, gross motor activities, visual motor activities, and balance tracking; and a networked computing device having a processing device and a memory device. In some cases, the memory device can store information that, when executed by the processing device, causes the processing device to:
(a) activate the motion sensing device;
(b) cause the motion sensing device to detect physical motion by a user in proximity to the motion sensing device;
(c) run an assessment of a user's physical capabilities, wherein the assessment process comprises:

the networked computing device causing a series of motor-sensory exercises to display to the user on a screen, wherein the series of motor-sensory exercises:

includes at least one movement-based activity to test each brain function from a group of brain functions, the group of brain functions including at least vestibular, cerebellum, motor planning and timing, directionality, fine motor, visual motor perception, and visual and auditory memory, and

has a desired user-executed outcome and a maximum score available for each of the at least one movement-based activities;

the networked computing device causing the motion sensing device to track and measure the user's movements during display of each of the at least one movement-based activities;

the networked computing device causing the motion sensing device to transmit raw data related to the user's tracked and measured movements to the networked computing device via a network, wherein the networked computing device compares the transmitted data from the user's tracked and measured movements to the desired user-executed outcomes;

assigning a corresponding assessment score to the user for each of the at least one movement-based activities, wherein the assessment scores are based on similarity of the user's tracked and measured movements to the desired user-executed outcomes, and wherein each movement-based activity's assessment score is comprised of at least two sub-scores, one for activities completed on each side of the user's body;

storing the assessment scores and sub-scores;

determining feedback displays for the user based on the user's tracked and measured movements, wherein the feedback displays alert the user of movement changes to make in real-time during each of the corresponding at least one movement-based activities;

the networked computing device causing the determined feedback displays to display to the user on the screen; and

upon completion by the user of each of the at least one movement-based activities, determining whether the assessment scores and sub-scores each meet or surpass a corresponding optimal score, the optimal scores being lower than the maximum scores available;

(d) determine a first series of interactive activities that targets the brain function associated with a first of the at least one movement-based activities having an assessment score or sub-score that did not meet its optimal score;

(e) cause a first training activity from the first series of interactive activities to display to the user on the screen, wherein the first training activity has a desired user-executed outcome and a maximum score available;

(f) cause the motion sensing device to track and measure the user's movements during display of the first training activity;

(g) cause the motion sensing device to collect and transmit raw data related to the user's tracked and measured movements to the networked computing device via the network;

(h) compare the transmitted data from the user's tracked and measured movements to the desired user-executed outcome;

assign a corresponding training score to the user, wherein the training score is based on similarity of the user's tracked and measured movements to the desired user-executed outcome;

store the training score;

determine a training feedback display for the user based on the user's tracked and measured movements, wherein the feedback display alerts the user of movement changes to make in real-time during the first training activity;

cause the determined training feedback display to display to the user on the screen;

upon completion by the user of the first training activity, determine whether the training score meets or surpasses an optimal score, the optimal score being lower than the maximum score available;

cause either the first training activity or a second training activity from the first series of interactive activities to display to the user on the screen, wherein:

the first training activity is displayed if the optimal score is not met or surpassed;

the second training activity is displayed if the optimal score is met or surpassed; and

the second training activity has a desired user-executed outcome and a maximum score available;

repeat steps (f) through (m) for the selected training activity;

proceed to repeat steps (f) through (o) in an iterative or recursive manner, with each training activity's optimal score acting as a threshold for a next iteration, until each optimal score is met or surpassed for all training activities of the first series of interactive activities; and

determine when the user successfully completes the first series of interactive activities by determining when each optimal score for all training activities of the first series of interactive activities have been met or surpassed;

wherein either (1) slow objects are displayed on the screen and move slowly from left to right, and fast objects are displayed on the screen and move quickly from right to left; or (2) slow objects are displayed on the screen and move slowly from right to left, and fast objects are displayed on the screen and move quickly from left to right.

In some embodiments, the processing device of the system of the first example embodiment can determine that the user has successfully completed the first series of interactive activities, and it may then cause the motion sensing device to display a second series of interactive activities to the user on the screen. Further, each training activity from the second series of interactive activities can have a desired user-executed outcome, an optimal score, and a maximum score available. The processing device can then proceed to repeat steps (f) through (o) in an iterative or recursive manner, with each training activity's optimal score acting as a threshold for a next iteration, until each optimal score is met or surpassed for all training activities of the second series of interactive activities. Lastly, the processing device can determine when the user successfully completes the second series of interactive activities by determining when each optimal score for all training activities of the second series of interactive activities have been met or surpassed.

In some embodiments, the first series of interactive activities can specifically instruct the user to use a left or a right side of the user's body, the first series of interactive activities can be displayed on a right or left side of the screen, the system can display the first series of activities in colors such as, but not limited to, navy, blue, purple, red, orange, yellow, and combinations of those colors, and the system can simultaneously play low or high frequency sounds during display of the first series of interactive activities. In some embodiments, slow objects can be displayed on the screen and move between an upper left portion and a bottom right portion of the screen. In other embodiments, slow objects can be displayed on the screen and move between an upper right portion and a bottom left portion of the screen.

In some embodiments, the networked computing device of the system of the first example embodiment can sync to Bluetooth-enabled glasses, the Bluetooth-enabled glasses being comprised of a left lens and a right lens, wherein a left half of the left lens and a left half of the right lens is red and a right half of the left lens and a right half of a right lens is blue. In some cases, the Bluetooth-enabled glasses are further comprised of earbuds enabled to play sound, such as the high and low frequency sounds described above.

The various embodiments described above are provided by way of illustration only and should not be construed to limit the claims attached hereto. Those skilled in the art will readily recognize various modifications and changes that may be made without following the example embodiments and applications illustrated and described herein and without departing from the true spirit and scope of the following claims.

We claim:

1. An exercise therapy video game and monitor system for physical targeted activation and cognitive skills for learning fluency of a user, the system comprising:
a first gaming platform networked to an interactive exercise software and configured to provide movement instructions to the user on a screen;
a motion sensing device including at least one sensor that is configured to track body positions and movements of the user and at least one processor configured to receive raw data from the at least one sensor and determine, corresponding to the body positions and movements of the user, when the user is replicating the movements of a first visual movement instruction displayed on the screen; and
a computing system networked to the interactive exercise software and configured to:
input the received raw data and evaluate the accuracy of the body positions and movements of the user that is replicating the movements of the first visual movement instruction, wherein values are generated that corresponds to the body positions and movements of the user;
provide a second visual movement instruction having a threshold level of difficulty, based on the generated values associated with the body positions and movements of the user and the first visual movement instruction, on the screen, wherein the second visual movement instructions instruct the user to put a first body part of the user in contact with a second body part of the user;
sense motion with the motion sensing device, and send motion data to the computing system for at least the position and movement associated with at least one of the first body part, the second body part, a third body part, and a fourth body part;
determine a value for velocity and acceleration associated with the at least one of the first, second, third, and fourth body parts based on the data;
confirm that the first body part is in contact with the second body part when the motion data associated with a first body part position and a second body part position indicates the first body part and the second body part are at least partially in the same position in space, and wherein a location value is assigned to the first and second body part location;
determine that the first, second, third, and fourth body parts ceased moving toward each other at approximately a same point in time;
display a score, based on the positions at a single point in time, of at least one of the first, second, third, and fourth body parts respectively, the velocity value, and the location value; and
assign a level of difficulty above or below the threshold level of difficulty for the second visual movement instruction based on the score.

2. The system of claim 1, wherein the first body part is a hand of the user and the second body part is a knee of the user.

3. The system of claim 2, wherein the third body part is an elbow of the user and the fourth body part is an ankle of the user.

4. The system of claim 3, wherein the hand is a right hand of the user, the knee is a left knee of the user, the elbow is a right elbow of the user, and the ankle is a left ankle of the user.

5. The system of claim 1, wherein the first body part is a finger of the user and the second body part is a nose of the user.

6. The system of claim 1, wherein if the first body part and the second body part do not appear in the same position in space, then the computing system concludes that the first body part is not in contact with the second body part.

7. The system of claim 1, wherein the second visual instruction is further based on a comparison of the received raw data associated with motion and position of each of the respective body parts compared to a desired user-executed outcome, wherein the desired user-executed outcome is based on the visual movement instruction; and wherein the computing device system further determines a score associated with the comparison.

8. The system of claim 1, wherein the computing system calculates the score by:
analyzing the raw data to determine an executed outcome for the body positions and movements of the user after a predetermined amount of time, wherein the executed outcome is the position of the first body part and the second body part when the position of the first body part was closest to the position of the second body part in space;
calculating a distance between the first body part position and the second body part position when they were closest to each other;
comparing the distance between the first body part position and the second body part position when they were closest to each other to a pre-determined accuracy score;
displaying the accuracy score comparison on the screen; and
updating the second visual movement instruction based on the accuracy score comparison.

9. The system of claim 8, wherein the accuracy score is 100 when the computing system determines that the first body part has contacted the second body part.

10. The system of claim 9, wherein the user obtains at least three scores for the second visual movement instruction above the predetermined threshold score, the level of difficulty is increased to the next higher level.

11. The system of claim 9, wherein the accuracy score is less than a pre-determined threshold when the computing system determines that the first body part has not made contact with the second body part.

12. The system of claim 1, further comprising a second gaming platform networked to the interactive exercise software and the computing system.

13. The system of claim 12, wherein the second gaming platform is a foot pressure sensitive device having a plurality of sensors that track foot positions and movements of the user and create raw data.

14. The system of claim 13, wherein the second gaming platform outputs raw data to the computing system.

15. The system of claim 14, wherein the computing system combines the raw data from the motion sensing device and the raw data from the second gaming platform to create a combined raw data set.

16. The system of claim 15, wherein the computing system uses the combined raw data set to:
evaluate the accuracy of the user's body and foot positions and movements; and
provide feedback to the user by displaying a comparison of the user's body and foot positions and movements to the visual movement instructions on the screen.

17. An exercise therapy video game and monitor system for physical targeted activation and cognitive skills for learning fluency of a user, the system comprising:
a first gaming platform networked to an interactive exercise software and configured to provide movement instructions to the user on a screen;
a motion sensing device including at least one sensor that is configured to track fine-body positions and movements of the user and at least one processor configured to receive raw data from the at least one sensor and determine, corresponding to the fine-body positions and movements of the user, when the user is replicating the movements of an assessment movement instruction displayed on the screen; and
a computing system networked to the interactive exercise software and configured to:
input the received raw data and evaluate the accuracy of the fine-body positions and movements of the user that is replicating the movements of the assessment movement instruction, wherein values are generated that corresponds to the fine-body positions and movements of the user;
provide a training movement instruction having a threshold level of difficulty, based on the generated values associated with the fine-body positions and movements of the user and the assessment movement instruction, on the screen, wherein the training movement instructions direct the user to put a first body part of the user in contact with a second body part of the user;
sense motion with the motion sensing device, and send motion data to the computing system for at least the position and movement associated with at least one of the first body part, the second body part, a third body part, and a fourth body part;
determine a value for velocity and acceleration associated with the at least one of the first, second, third, and fourth body parts based on the motion data;
confirm that the first body part is in contact with the second body part when the motion data associated with a first body part position and a second body part position indicates the first body part and the second body part are at least partially in the same position in space, and wherein a location value is assigned to the first and second body part location;
determine that the first, second, third, and fourth body parts ceased moving toward each other at approximately a same point in time;
display a score, based on the positions at a single point in time, of at least one of the first, second, third, and fourth body parts respectively, the velocity value, and the location value; and
assign a level of difficulty above or below the threshold level of difficulty for the training movement instruction based on the score.

18. The system of claim 17, wherein the interactive exercise software further instructs the user to replicate the training movement instruction at least three times where the score is greater than the threshold score for the level of difficulty.

19. The system of claim 18, wherein the interactive exercise software further instructs the user to replicate the training movement instruction of a higher level of difficulty once the user has received at least three scores higher than the threshold score of the current level of difficulty.
